(19) United States
(12) Patent Application Publication
(10) Pub. No.: US 2002/0177575 A1
(43) Pub. Date: Nov. 28, 2002

(54) Identification of farnesyl-protein transferase as a target for systemic lupus erythematosus therapies

(76) Inventors: Ward Wakeland, Dallas, TX (US); Kui Liu, Plano, TX (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(21) Appl. No.: 10/138,724

(22) Filed: May 3, 2002

Related U.S. Application Data
(60) Provisional application No. 60/288,868, filed on May 4, 2001.

(51) Int. Cl. A61K 48/00; A61K 31/517; A61K 31/495; A61K 31/407; A61K 31/401

(52) U.S. Cl. 514/44; 514/141; 514/266.3; 514/312; 514/255.02; 514/396; 514/423; 514/416; 514/411; 514/456

(57) ABSTRACT

The invention relates to the identification of farnesyl-protein transferase (FPT) as a gene that is upregulated in systemic lupus erythematosus (SLE). Given the widespread availability of inhibitors of FPT, the inventors propose to treat the symptoms of SLE using such inhibitors.

### BACKGROUND OF THE INVENTION

This application claims benefit of priority to U.S. Provisional Serial No. 60/288,868, filed on May 4, 2001, the entire contents of which are incorporated by reference herein. The federal government owns certain rights in this application by virtue of grant support for the National Institutes of Health (NIH A1-39824).

1. Field of the Invention

The present invention relates generally to the fields of molecular biology and pathology. More particularly, it concerns the identification of genes involved in the breakdown of immune tolerance leading to the development of systemic autoimmunity, particularly systemic lupus erythematosus (SLE), and methods of treating SLE based thereon.

2. Description of Related Art

Systemic lupus erythematosus (SLE) is mediated by a complex interaction of genetic and environmental elements. Although the crucial role of genetic predisposition in susceptibility to SLE has been known for decades, only minimal progress has been made towards elucidating the specific genes involved in human disease. Recently, several groups have reported linkage analyses that provide estimates of the number and chromosomal locations of at least some of the susceptibility genes for human SLE. These studies have provided interesting insights into the complexity of the genetic interactions involved in SLE but have not resulted in the identification of specific genes or genetic pathways involved in disease pathogenesis. This reflects the difficulties inherent in the analysis of complex genetic diseases, coupled with the absence of sample repositories of sufficient size to allow definitive genetic analysis.

Genetic analyses of lupus susceptibility in the mouse have progressed significantly over the last 5 years, predominantly due to the availability of unique and powerful experimental genetic tools. The chromosomal locations of genes mediating susceptibility in the NZB/W, MRL/lpr, and BXSB mouse models have been determined via genome scans. These studies have demonstrated that susceptibility to lupus in the mouse is inherited in a complex fashion that is quite similar to human SLE, involving both genetic interactions and additive effects of individual genes.

In previous studies, the inventors' laboratory has segregated three different gene clusters from the NZM2410 lupus-prone mouse. These clusters, designated Sle1, Sle2 and Sle3, were introgressed individually into B6 inbred mice. Wakeland et al. (1997). When recombined with either Sle2 or Sle3, the Sle1 locus causes fatal lupus nephritis Mohan et al. (1999). Thus, one or more gene within the Sle1 gene cluster (CRP, DEDD, USF-1, SLAM, CD48, Ly-9 and 2B4) play an essential role in disease pathogenesis in both human and mouse systemic autoimmunity. It remains, however, to identify appropriate points of therapeutic intervention.

### SUMMARY OF THE INVENTION

Thus, in accordance with the present invention, there is provided a method for treating systemic lupus erythematosus (SLE) comprising administering to a subject suffering from SLE a first inhibitor of farnesyl-protein transferase (FPT). The inhibitor may be administered through intravenous, intraarterial, intramuscular, intraperitonical, intradermal, intranasal, oral or topical routes. It may be a pharmaceutical compound, such as a tricyclic, a quinolinone, a quinazolinone, a benzopyranone, an imidazole, a benzo(F)isoindole, a thioproline, a biheteroaryl, a piperazinyl, or a piperazinonyl. It may be a farnesyl pyrophosphate analogue or a peptide/peptidomimetic, for example, containing or emulating the CAAX-box motif.

Alternatively, the inhibitor may be a nucleic acid encoding an FPT antisense molecule or an FPT ribozyme under the control of a promoter active in the subject. The nucleic acid may be contained in a vector, for example, a viral vector such as an adenoviral vector, an adeno-associated viral vector, a retroviral vector, a vaccinia viral vector, a herpesviral vector or a polyoma viral vector. The antisense molecule may target an FPT promoter, intron, transcription start site, translation start site, splice junction or coding region. The inhibitor may also be encapsulated in a liposome.

The inhibitor may be administered at least a second time, for example, as part of a continuous maintenance drug regimen. The subject may be monitored for toxic effects following administration of the inhibitor, for example, where cells from the subject are monitored for FPT activity following administration of the inhibitor. The method may further comprise administering to the subject a second inhibitor of FPT distinct from the first inhibitor. The method may also comprise administering to the subject a conventional SLE therapeutic compound. The conventional SLE therapeutic compound may be administered prior to, after or at the same time as the inhibitor. The conventional SLE therapeutic compound may be administered at least a second time, for example, as part of a continuous maintenance drug regimen.

The conventional SLE therapeutic compound may be selected from the group consisting of a non-steroidal anti-inflammatory drug (NSAID), an antimalarial drug, a corticosteroid hormone and an immunosuppressive drug. The NSAID may be selected from the group consisting of ibuprofen, naproxen, sulindac, diclofenac, piroxicam, ketoprofen, diflunisal, nabumetone, etodolac, oxaprozin and indomethacin. The antimalarial drug may be selected from the group consisting of hydrochloroquine, chloroquine and quinacrine. The corticosteroid hormone may be selected from the group consisting of prednisone, hydrocortisone, methylprednisone and dexamethasone. The immunosuppressive agent may be selected from the group consisting of azathioprine, cyclophosphamide, and methotrexate. The subject may also be monitored for toxic effects following administration of the conventional SLE therapeutic compound.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Expression of CAAX box farnesyl-protein transferase alpha chain gene in purified splenic B lymphocytes from female mice at 4 months of age. Each congenic strain was compared to a B6 control strain. For each line, the left end represents hybridization intensity for B6 while the right end represents hybridization intensity for Sle1-congenic strain. The number on the top represents the mean of ratios.

FIGS. 2A & 2B: Sle1 mediates dysregulation of ERK MAP kinase pathway in B lymphocytes. FIG. 2A—Immunoblotting of the phosphorylated (activated) form of ERK1 and ERK2 as well as total ERK1 and ERK2. These results demonstrate an increase in the activated form of ERK2 in B lymphocytes from B6.Sle1(a+b) mice. FIG. 2B—Spleen weights for the mice that were analyzed.

FIG. 3: Inhibitory effect of perillyl alcohol (POH) on the production of antinuclear autoantibodies in B6.Sle1(a+b) mice. T0 represents the OD value for measuring the titer of anti-Histone/DNA antibodies before treatment. T represents the OD value for measuring the titer of anti-Histone/DNA antibodies at a time point during or after treatment. Each point represents Mean±SE. Mice in control group were given vehicle (Tricaprylin) only.

### DESCRIPTION OF ILLUSTRATIVE EMBODIMENTS

Although it is estimated to affect one out of every 1,000 white persons, and one out of every 250 black women from 18 to 65 years of age, systemic lupus erythematosus (SLE) is certainly not the most common example of autoimmune disease. Positive antinuclear antibodies are extremely common in the general population, occurring in as many as 10 to 20 percent of young women. Localized autoimmune disorders, such as autoimmune thyroid disease, are also much more common than systemic lupus erythematosus. However, because systemic lupus erythematosus is a chronic disease, patients require extensive health education in terms of their responsibility in managing their condition. This requires compliance with office visits and medications, and lifestyle modifications to reduce or prevent associated problems such as hyperlipidemia, obesity and hypertension. More troublesome is the lack of any cure—therapies are limited to alleviating symptoms.

The present inventors have focused their efforts on identifying molecular changes that occur in the cells of subjects afflicted with SLE. Making use of animal models for the disease, these studies have identified multiple loci (Sle1, Sle2, Sle3), mutations in which combine to produce SLE-type pathologies. From studies on these animals, a number of different candidate genes have been identified as effectors of the SLE phenotype. In particular, it now has been shown that one specific gene is upregulated in affected cells, providing a point of intervention that may be the first truly curative therapeutic approach for this disease.

A. The Present Invention

In order to identify molecular mechanisms by which Sle1 mediates anti-nuclear autoantibody production, the inventors performed microarray analysis to compare the gene expression profiles of B lymphocytes from C57BL/6 and Sle1-congenic strains. The microarray constructed consisted of about 11,800 cDNA clones. The analysis indicated that expression of farnesyl-protein transferase (FPT) alpha chain gene is upregulated in B6.Sle1 and B6.Sle1(a+b) B lymphocytes. Since farnesyl-protein transferase catalyzes the posttranslational farnesylation of Ras protein, the upregulated expression of this gene indicated the dysregulation of Ras-related signaling pathway.

This observation also is consistent with the inventors' finding that expression of c-myc is upregulated in B6.Sle1 and B6.Sle1(a+b) B lymphocytes, and correlates with splenomegaly as well as anti-nuclear autoantibody (ANA) production. Because FPT is a critical regulator of Ras signaling pathway, inhibition of its activity by FPT inhibitors should downregulate Ras-related B cell activation signaling, and lead to the suppression of ANA production. Thus, according to the present invention, inhibition of FPT is proposed as therapeutic strategy for SLE.

B. Systemic Lupus Erythematosus

(i) SLE

Systemic Lupus Erythematosus is a type of autoimmune disease. It causes inflammation and damage of various body tissues, including the joints, skin, kidneys, heart, lungs, blood vessels and brain. Although people with the disease may have many different symptoms, some of the most common ones include extreme fatigue, painful or swollen joints (arthritis), unexplained fever, skin rashes, and kidney problems. SLE is also known as a rheumatic disease. The rheumatic diseases are a group of disorders that cause aches, pain, and stiffness in the joints, muscles, and bones.

Each person's experience with lupus is different. Symptoms can range from mild to severe and may come and go over time. In addition to the typical characteristics listed above, subjects also complain of chest pain, hair loss, sensitivity to the sun, anemia, and pale or purple fingers and toes from cold and stress. Some people also experience headaches, dizziness, depression, or seizures. New symptoms may continue to appear years after the initial diagnosis, and different symptoms can occur at different times.

In some people with lupus, only one system of the body such as the skin or joints is affected. Other people experience symptoms in many parts of their body. Just how seriously a body system is affected also varies from person to person. Most commonly, joints and muscles are affected, causing arthritis and muscle pain. Skin rashes also are quite common. The following systems in the body also can be affected by SLE.

Kidneys: Inflammation of the kidneys (nephritis) can impair their ability to effectively get rid of waste products and other toxins from the body. Because the kidneys are so important to overall health, lupus in the kidneys generally requires intensive drug treatment to prevent permanent damage. There is usually no pain associated with kidney involvement, although some patients may notice that their ankles swell. Most often the only indication of kidney disease is an abnormal urine test.

Central nervous system: In some patients, lupus affects the brain or central nervous system. This can cause headaches, dizziness, memory disturbances, vision problems, stroke, or changes in behavior. Some of these symptoms, however, also can be caused by some treatments of lupus or by the emotional stress of dealing with the disease.

Blood vessels: Blood vessels may become inflamed (vasculitis), affecting the way blood circulates through the body. The inflammation may be mild, and may not require treatment.

Blood: People with lupus may develop anemia or leukopenia (a decreased number of white blood cells). Lupus also may cause thrombocytopenia, a decreased number of platelets in the blood that contributes to an increased chance of bleeding. Some people with lupus may have an increased risk for blood clots.

Lungs: Some people with lupus develop pleuritis, an inflammation of the lining of the chest cavity that causes chest pain, particularly with breathing. Patients with lupus also may get pneumonia.

Heart: In some people with lupus, inflammation can occur in the arteries that supply blood to the heart (coronary vasculitis), the heart itself (myocarditis and endocarditis), or the membrane that surrounds it (pericarditis), causing chest pains or other symptoms.

The cause of SLE is unknown. It is likely that there is no single cause but rather a combination of genetic, environmental, and possibly hormonal factors that work together to cause the disease. The exact cause may differ from one person to another. Research suggests that genetics plays an important role; however, no specific “lupus gene” has been identified. Instead, it appears that several genes may increase a person's susceptibility to the disease.

The fact that lupus can run in families indicates that development of this disease has a genetic basis. In addition, studies of identical twins have shown that lupus is much more likely to affect both members of a pair of identical twins who share the exact same set of genes than two nonidentical twins or other siblings. Because the risk for identical twins is far less than 100 percent, however, scientists believe that genes alone cannot account for who contracts SLE. Thus, other factors must also play a role. Some of the factors that scientists are studying include sunlight, stress, certain drugs, and infectious agents such as viruses. Even though a virus might trigger the disease in susceptible individuals, a person cannot “catch” lupus from someone else.

At present, there is no cure for lupus. However, the symptoms of lupus can be controlled with appropriate treatment, and most people with the disease can lead active, healthy lives. Lupus is characterized by periods of illness, called flares, and periods of wellness, or remission. Understanding how to prevent flares and how to treat them when they do occur helps people with lupus maintain better health.

(ii) Sle Loci

Data indicates that three separate genetic pathways interact during the development of severe lupus nephritis in this mouse model. During the first stage, genes such as Sle1 trigger the loss of immune tolerance to nuclear autoantigens and mediate the initiation of autoimmunity (Morel et al., 1996; Mohan et al., 1998). Genes in this pathway are capable of causing the initiation of a humoral autoimmune response to nuclear antigens; however, this response is not pathogenic in the absence of genes in the other pathways. In this regard, many first-degree relatives of SLE probands exhibit a similar seropositive phenotype without severe disease pathogenesis (Winchester, 1992). The second genetic pathway mediating lupus susceptibility contains genes causing generalized immune hyper-responsiveness or dysregulation. Genes such as Sle2, Sle3, lpr, gld and Yaa would all be included in this pathway. These genes often do not generate autoimmune phenotypes in lupus-resistant genomes, but strongly enhance the expansion of the autoimmune response when combined with genes that mediate the loss of tolerance to nuclear autoantigens (Mohan et al., 1997). The final class of lupus susceptibility genes are those that potentiate end organ damage. Theoretically, end organ damage could be enhanced by a variety of molecular mechanisms, including genes that modify immune effector functions (such as Fc receptors (Clynes et al., 1998)) and those that modify the end organ itself.

Based upon an analysis of the epistatic interactions that occur when SLE susceptibility genes are re-combined on the B6 background, fatal lupus nephritis is produced when Sle1 is combined with either Sle2 or Sle3 on the non-autoimmune B6 genome (Mohan et al., 1999). This is the first example of the successful reconstruction of a complex disease phenotype on a normal genome via the reassembly of congenic intervals. A key aspect of this finding is the observation that two genes in combination are sufficient to generate fatal lupus nephritis. This observation makes feasible the unambiguous identification of the disease genes in each of these intervals via assaying their capacity to elicit fatal lupus when combined with the genes in a second congenic strain. For example, the gene(s) in the Sle1 interval that is essential for disease will produce fatal lupus when they are bred with B6.NZMc7 (which carries the intact Sle3 interval).

Congenic strain construction is a requisite initial stage in identifying the genes causing murine lupus. Each congenic strain becomes a monogenic model for a specific component phenotype associated with the disease process. A detailed analysis of the phenotypes expressed by these recombinants reveals several important findings about Sle1. First, Sle1 is not a single gene, but is instead a gene cluster containing at least 4 genes that impact susceptibility to autoimmune lupus nephritis. All four genes, which were designated Sle1a-Sle1d, have been isolated on truncated congenic sub-intervals. Phenotypic analyses of these subintervals indicate that each gene in the Sle1 cluster expresses a unique subset of the multiple component phenotypes associated with the Sle1 interval.

Two features of the genes in the Sle1 cluster suggest that they may have closely-related functions. First, both Sle1a and Sle1b break tolerance to nuclear chromatin in a manner resulting in the preferential production of IgG autoantibodies against H2A/H2B-DNA subnucleosome antigens. A second intriguing feature of this system is the recent observation that Sles1, a locus-specific suppressive modifier located on chromosome 17 in the NZW genome, can suppress all the autoimmune phenotypes produced by the entire Sle1 cluster (Morel et al., 1999). The ability of Sles1 to suppress all of the component phenotypes associated uniquely with Sle1a, Sle1b, and Sle1d suggests that all these genes function within a single biologic pathway that mediates a breach in tolerance to chromatin autoantigens.

The results of several recent linkage studies of SLE in humans reveal an intriguing relationship between the genetics of susceptibility in humans and mice. The region of the human genome that is syntenic with the interval containing the murine Sle1 gene contains a cluster of genes associated with human SLE susceptibility (Tsao et al., 1997; Harley et al., 1998; Moser et al., 1998; Gaffney et al., 1998; Shai et al., 1999). The precise positions within this interval that are most strongly associated with human SLE susceptibility have varied somewhat in the findings of different groups, and in some cases multiple peaks have been detected in the 1q23 to 1q42 region. The Sle1 gene cluster is located within a region of murine chromosome 1 that is syntenic with the 1q23 to 1q42 region in humans, strongly supporting the contention that the human homologue of the Sle1 gene cluster is a key genetic predisposing factor for human SLE. In this regard, the distance between CRP and ADPRT in humans is much longer (˜55 cM) than the distance from Crp to Adprp in mouse (˜8 cM), suggesting that synteny within this region may not be complete. Nonetheless, this region remains a strong example of homologous mapping of disease susceptibility genes in humans and mice.

C. SLE Therapy

The present invention is directed to the use of FPT inhibitors in the treatment of SLE. By “treatment,” the inventors mean that symptoms of SLE will be lessened to an extent which benefits the patient. Such benefit may be increased mobility or energy, increased ability to undertake normal daily activities, and in particular, decreased pain. However, given the nature of the treatment, it is conceivable that such therapies could effectively cure SLE by eliminating all of the symptoms associated with the disease.

(i) Pharmaceutical Inhibitors

The following U.S. Patents, disclosing inhibitors of FPT, each are specifically incorporated by reference:

U.S. Pat. Nos. 6,225,322, 6,218,406; 6,218,401, 6,214,828, 6,211,193, 6,187,786, 6,177,432, 6,169,096, 6,159,984, 6,143,766, 6,143,758, 6,127,366, 6,124,465, 6,124,295, 6,103,723, 6,093,737, 6,090,948, 6,080,870, 6,077,853, 6,071,907, 6,066,738, 6,063,930, 6,060,038, 6,054,466, 6,051,582, 6,051,574, 6,051,565, 6,040,305, 6,037,350, 6,030,982, 6,028,201, 6,015,817, 6,013,662, 6,011,029, 6,001,835, 5,998,407, 5,990,277, 5,985,879, 5,981,562, 5,977,134, 5,976,851, 5,972,984, 5,972,966, 5,972,942, 5,968,965, 5,968,952, 5,965,578, 5,965,570, 5,958,940, 5,945,430, 5,945,429, 5,939,557, 5,939,439, 5,936,097, 5,932,590, 5,929,077, 5,925,651, 5,925,648, 5,922,883, 5,919,785, 5,919,780, 5,914,341, 5,891,889, 5,889,053, 5,885,995, 5,883,105, 5,880,140, 5,874,452, 5,872,136, 5,872,135, 5,869,682, 5,861,395, 5,859,015, 5,859,012, 5,856,439, 5,856,326, 5,854,265, 5,854,264, 5,852,010, 5,817,678, 5,789,438, 5,780,492, 5,780,488, 5,756,528, 5,753,650, 5,750,567, 5,736,539, 5,710,171, 5,703,241, 5,703,067, 5,686,472, 5,663,193, 5,661,161, 5,661,128, 5,652,257, 5,631,280, 5,627,202, 5,627,057, 5,624,936, 5,585,359, 5,578,629, 5,576,313, 5,576,293, 5,571,835, 5,567,729, 5,536,750, 5,534,537, 5,523,456, 5,523,430, 5,510,510, 5,510,371, 5,504,212, 5,504,115, 5,491,164, 5,480,893, 5,468,733, 5,439,918, 5,436,263, 5,420,334, 5,420,245, 5,420,157, 5,352,705, 5,350,867, 5,340,828, 5,326,773, 5,260,479, 5,260,465, 5,245,061, 5,238,922, 5,185,248

Each of the inhibitors disclosed may be used, according to the present invention, to treat SLE. Specific information on how to make, formulate and administer these inhibitors are provided in the incorporated references, as well as in the sections below.

(ii) Genetic Inhibitors

In alternative embodiments, FPT nucleic acids may be used to create antisense constructs that hybridize, under intracellular conditions, to the DNA or mRNA for FPT. The term “antisense construct” is intended to refer to nucleic acids, preferably oligonucleotides, that are complementary to the base sequences of a target DNA or RNA. Targeting double-stranded (ds) DNA with an antisense construct leads to triple-helix formation; targeting RNA will lead to double-helix formation. Antisense nucleic acids, when introduced into a target cell, specifically bind to their target polynucleotide and interfere with transcription, RNA processing, transport, translation and/or stability. Antisense RNA constructs, or DNA encoding such antisense RNAs, may be employed to inhibit FPT gene transcription or translation or both within the cells of the present invention. Nucleic acids encoding FPT can be found in U.S. Pat. No. 5,976,851.

Antisense constructs may be designed to bind to the promoter and other control regions, exons, introns or even exon-intron boundaries of a gene. Antisense RNA constructs, or DNA encoding such antisense RNA's, may be employed to inhibit gene transcription or translation or both within a host cell, either in vitro or in vivo, such as within a host animal, including a human subject. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine (A:T), in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

As used herein, the term “complementary” means nucleic acid sequences that are substantially complementary over their entire length and have very few base mismatches. For example, nucleic acid sequences of fifteen bases in length may be termed complementary when they have a complementary nucleotide at thirteen or fourteen positions with only a single mismatch. Naturally, nucleic acid sequences which are “completely complementary” will be nucleic acid sequences which are entirely complementary throughout their entire length and have no base mismatches.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

While all or part of the FPT gene sequences may be employed in the context of antisense construction, short oligonucleotides are easier to make and increase in vivo accessibility. However, both binding affinity and sequence specificity of an antisense oligonucleotide to its complementary target increases with increasing length. One can readily determine whether a given antisense nucleic acid is effective at targeting of the corresponding host cell gene simply by testing the constructs in vitro to determine whether the function of the endogenous gene is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression.

Another method for inhibiting FPT expression contemplated in the present invention is via ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Several different ribozyme motifs have been described with RNA cleavage activity (Symons, 1992). Examples that are expected to function equivalently for the down regulation of FPT include sequences from the Group I self splicing introns including Tobacco Ringspot Virus (Prody et al., 1986), Avocado Sunblotch Viroid (Palukaitis et al., 1979; Symons, 1981), and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al., 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in FPT targeted ribozymes is simply a matter of preparing and testing a given sequence, and is a routinely practiced “screening” method known to those of skill in the art.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

1. DNA Delivery Using Viral Vectors

The ability of certain viruses to infect cells or enter cells via receptor-mediated endocytosis, and to integrate into host cell genome and express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

a. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The El region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) discloses improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al, 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

b. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Clark et al., 1995). Cell lines carrying the rAAV DNA as an integrated provirus can also be used (Flotte et al., 1995).

C. Retroviral Vectors Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

d. Other Viral Vectors

Other viral vectors may be employed as expression constructs in the present invention. Vectors derived from viruses such as vaccinia virus (Ridgeway, 1988; Coupar et al., 1988), sindbis virus, cytomegalovirus and herpes simplex virus may be employed. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

e. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Other Methods of DNA Delivery

In various embodiments of the invention, DNA is delivered to a cell as an expression construct. In order to effect expression of a gene construct, the expression construct must be delivered into a cell. As described herein, the preferred mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane. Some of these techniques may be successfully adapted for in vivo or ex vivo use, as discussed below.

a. Liposome-Mediated Transfection

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

b. Particle Bombardment

Another embodiment of the invention for transferring a naked DNA expression construct into cells may involve particle bombardment, which can be useful in topical applications. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

C. Receptor Mediated Transfection

Still further expression constructs that may be employed to deliver the expression construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of other mammalian cell types is described by Wu and Wu (1993) and Curiel (1994).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0 273 085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

(iii) Combination Treatments

In order to increase the effectiveness of the pharmaceutical or genetic therapies described above, it may be desirable to combine these compositions with other agents effective in the treatment of SLE, such as immunosuppressive agents. These compositions are provided in a combined amount effective to alleviate or reduce some symptom of SLE. This process may involve contacting the cells of a patient with the FPT inhibitors of the present invention and the other agent(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Alternatively, the non-FPT therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, FPT inhibitor is “A” and the other agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Several types of drugs currently are used to treat lupus, as discussed below. Each of these drugs may be used in the combination therapies described above.

For SLE patients with joint pain, fever and swelling, drugs that decrease inflammation, referred to as nonsteroidal anti-inflammatory drugs (NSAIDs), are often used. Common side effects of NSAIDs, including those available over the counter, can include stomach upset, heartburn, diarrhea, and fluid retention. Some lupus patients also develop liver and kidney inflammation while taking NSAIDs, making it especially important to stay in close contact with the doctor while taking these medications. A list of NSAIDs used to treat SLE are: ibuprofen, naproxen, sulindac, diclofenac, piroxicam, ketoprofen, diflunisal, nabumetone, etodolac, oxaprozin and indomethacin.

Antimalarials also are another type of drug commonly used to treat lupus. These drugs were originally used to treat the symptoms of malaria, but doctors have found that they also are useful treatments for lupus. Exactly how antimalarials work in lupus is unclear, but scientists believe that they may work by suppressing parts of the immune response. Specific antimalarials used to treat lupus include hydrochloroquine, chloroquine and quinacrine. Continuous treatment with antimalarials may prevent flares from recurring. Side effects of antimalarials can include stomach upset and, extremely rarely, damage to the retina of the eye.

The mainstay of lupus treatment involves the use of corticosteroid hormones, such as prednisone, hydrocortisone, methylprednisolone and dexamethasone. Corticosteroids are related to cortisol, which is a natural anti-inflammatory hormone. They work by rapidly suppressing inflammation. Corticosteroids can be given by mouth, in creams applied to the skin, or by injection. Because they are potent drugs, the doctor will seek the lowest dose with the greatest benefit. Short-term side effects of corticosteroids include swelling, increased appetite, weight gain, and emotional ups and downs. These side effects generally stop when the drug is stopped. It can be dangerous to stop taking corticosteroids suddenly, so it is very important that the doctor and patient work together in changing the corticosteroid dose. Sometimes doctors give very large amounts of corticosteroid by vein (“bolus” or “pulse” therapy). With this treatment, the typical side effects are less likely and slow withdrawal is unnecessary.

Long-term side effects of corticosteroids can include stretch marks on the skin, excessive hair growth, weakened or damaged bones, high blood pressure, damage to the arteries, high blood sugar, infections, and cataracts. Typically, the higher the dose of corticosteroids, the more severe the side effects. Also, the longer they are taken, the greater the risk of side effects. Researchers are working to develop alternative strategies to limit or offset the use of corticosteroids. For example, corticosteroids may be used in combination with other, less potent drugs, or the doctor may try to slowly decrease the dose once the disease is under control. People with lupus who are using corticosteroids should talk to their doctors about taking supplemental calcium and vitamin D to reduce the risk of osteoporosis.

For patients whose kidneys or central nervous systems are affected by lupus, a type of drug called an immunosuppressive may be used. Immunosuppressives, such as azathioprine and cyclophosphamide, restrain the overactive immune system by blocking the production of some immune cells and curbing the action of others. These drugs may be given by mouth or by infusion. Side effects may include nausea, vomiting, hair loss, bladder problems, decreased fertility, and increased risk of cancer and infection. The risk for side effects increases with the length of treatment. As with other treatments for lupus, there is a risk of relapse after the immunosuppressives have been stopped.

In special circumstances, patients may require stronger drugs to combat the symptoms of lupus. For patients who cannot take corticosteroids, a type of immunosuppressive drug called methotrexate may be used to help control the disease. Patients who have many body systems affected by the disease may receive intravenous gamma globulin, a blood protein that increases immunity and helps fight infection. Gamma globulin also may be used to control acute bleeding in patients with thrombocytopenia or to prepare a person with lupus for surgery.

D. Pharmaceutical Compositions

(i) Pharmaceutically Acceptable Carriers

Aqueous compositions of FPT inhibitors of the present invention comprise are dissolved or dispersed in a pharmaceutically acceptable carrier or aqueous medium. Aqueous compositions of gene therapy vectors expressing any of the foregoing are also contemplated. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, subcutaneous, or even intraperitoneal routes. The preparation of an aqueous composition that contains an inhibitor of FPT as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An inhibitor of FPT can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The FPT inhibitors may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

(ii) Liposomes and Nanocapsules

In certain embodiments, the use of liposomes and/or nanoparticles is contemplated for the delivery of FPT inhibitors. The formation and use of liposomes is generally known to those of skill in the art, and is also described below.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The following information may also be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

(i) Congenic Dissection

Congenic dissection is a strategy in which each gene contributing to a polygenic disease (such as lupus) is segregated into an individual sub-strain of an inbred mouse strain. The inventors' laboratory previously conducted congenic dissection of the lupus-prone NZM2410 mouse by introgression of three susceptibility intervals onto the non-autoimmune B6 inbred strain. This process created a collection of B6-congenic strains, each carrying a specific lupus-susceptibility gene in a genomic interval derived from NZM2410. Analyses of the immunologic phenotypes expressed by each congenic strain can then be used to identify the specific component phenotype that each contributes to the development of fatal autoimmune lupus nephritis in NZM2410 (Morel et al., 1996; Wakeland et al. 1997; Morel et al., 1997; Mohan et al., 1998; Morel et al., 1999; Morel et al., 1999).

(ii) Phenotypic Analysis with Gene Expression Microarrays.

Gene expression microarray analysis (Glynne et al., 1999; Bowtell et al., 1999) can provide insights into the genetic pathways that each Sle susceptibility interval dysregulates and to provide important insights into the molecular mechanisms that are impaired by individual susceptibility genes. Also, gene expression microarray analysis can be coupled with congenic recombinant analysis to identify epistatic genetic modifiers influencing specific genetic pathways in lymphocytes. Early results with small murine gene expression microarrays (1200 genes, Clontech) demonstrated consistent variations in the gene expression patterns between normal B6 and B6.NZM congenic mice. For example, B lymphocytes and CD4+ T cells from B6.Sle1 mice up-regulate the expression of a specific isoform of c-myc and other genes at two months of age, well prior to the initiation of autoantibody production.

These results establish the feasibility of using gene expression microarrays to detect and characterize the molecular pathways that are modified by specific lupus susceptibility alleles. The data indicate that both T and B cells are activated in B6.Sle1 mice well prior to the initiation of ANA production and that this activation is being triggered via a pathway that activates the transcription of a specific isoform of c-myc. This finding correlates with those of other groups who have previously reported that c-myc overexpression correlates with the production of autoantibody (Boumpas et al., 1986). It further suggests that endogenous and exogenous factors which lead to the expression of autoimmunity might share the induction of proto-oncogene expression as a common pathogenic step with Sle1. Since c-myc plays a vital role in cell-cycle progression, deregulated expression of c-myc can overcome cell-cycle arrest and promote cellular proliferation. In the case of CD4+T and B lymphocytes, which are frequently renewed, overexpression of c-myc could cause the dysregulation of their proliferation and, consequently, the survival of autoreactive CD4+T and B cells.

(iii) Farnesyl-Protein Transferase is a Potential Therapeutic Target for Lupus

In order to identify the molecular mechanisms by which Sle1 mediates antinuclear autoantibody production, the inventors performed microarray analysis to compare the gene expression profiles of B lymphocytes from C57BL/6 and Sle1-congenic strains. The microarray constructed consisted of about 5600 sequence-verified cDNA clones from Research Genetics and 6200 IMAGE Consortium mouse cDNA clones (1536 clones from B cell line, 1920 clones from T cell line, 768 clones from thymus, 768 clones from macrophage cell line, and 1152 clones from spleen) from Incyte Genomics.

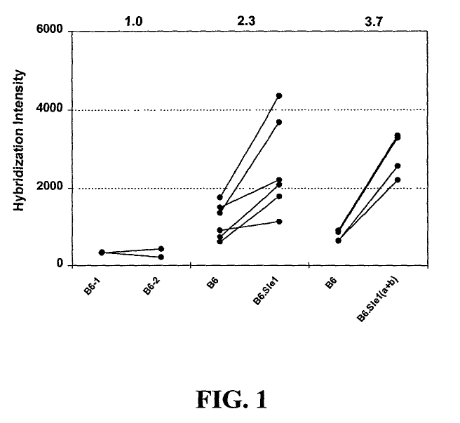

Total RNA isolated from purified splenic B lymphocytes was used to generate cDNA probes. Cy5-(for B6 control strain) and Cy3-(for Sle1-congenic strain) labeled probes were hybridized on the same array, and the image was collected and analyzed by using GenePix software from Axon. Analyses indicated that expression of FPT alpha chain gene is upregulated in B6.Sle1 and B6.Slel(a+b) B lymphocytes (FIG. 1). Since FPT catalyzes the post-translational farnesylation of Ras protein, the upregulated expression of this gene indicated the dysregulation of Ras-related signaling pathway. This observation also is consistent with the inventors' finding that expression of c-myc is upregulated in B6.Sle1 and B6.Sle1(a+b) B lymphocytes, and correlates with splenomegaly as well as anti-nuclear autoantibody (ANA) production. In addition, introgression of Sles1, a genetic modifier that specifically down-regulates the autoimmunity mediated by Sle1, leads to a downregulation of FPT.

(iv) Dysregulation in the Ras Pathway of Lupus Model Mice

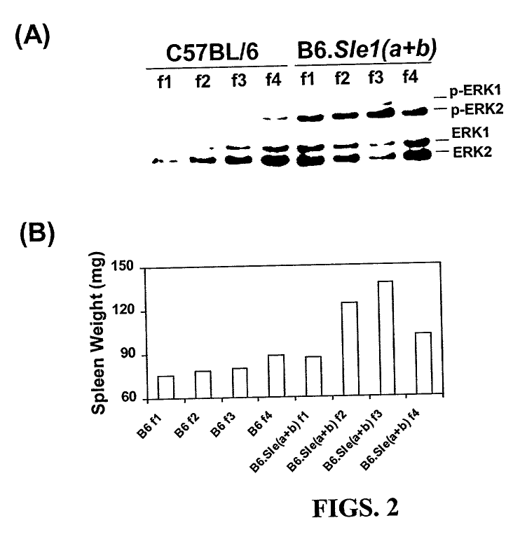

Activation of ras proteins initiates a phosphorylation cascade through sequential activation events of Raf, MEK, and ERK. The inventors' studies show that ERK2 MAP kinase is more activated in B lymphocytes isolated from B6.Sle1 mice (FIG. 2). This observation indicates that a dysregulation of the ras pathway is an intrinsic feature of the initial stage of autoimmunity in B6.Sle1 mice.

(v) Suppression of Anti-Nuclear Autoantibody Production Through Inhibition of Ras Protein Farnesylation

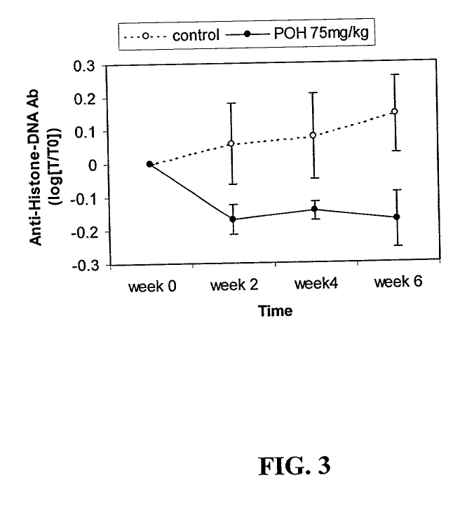

Perillyl alcohol (POH) is a monoterpene isolated from the essential oils of various plants, and has been shown to inhibit ras protein farnesylation. To study whether POH can inhibit anti-nuclear autoantibody (ANA) production, the inventors treated B6.Sle1(a+b) mice by daily intraperitoneal injection of POH at a dose of 75 mg/kg body weight for 4 weeks. Sera were collected before, during, and after treatment. Levels of anti-histone-DNA ANA were determined by ELISA. The results, shown in FIG. 3, indicate that Sle1-mediated ANA production can be suppressed by POH treatment.

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### F. REFERENCES

The following references, to the extent that they provide exemplary procedural or other details supplementary to those set forth herein, are specifically incorporated herein by reference.

Berzal-Herranz et al., Genes and Devel., 6:129-134, 1992.

Boumpas et al., Arthritis Rheum 29:755-760, 1986.

Bowtell et al., Nat Genet 21:25-32, 1999.

Cech et al., Cell, 27:487-496, 1981.

Chalfie et al., Science, 263:802-805, 1994.

Chang et al., Hepatology, 14:134A, 1991.

Chowrira et al., Biochemistry 32:1088-95, 1993.

Chowrira et al., J. Biol. Chem., 269:25856-25864, 1994.

Clark et al., Human Gene Therapy, 6:1329-1341, 1995.

Clynes et al., Science 279:1052-1054, 1998.

Coffin, “Retroviridae and their replication,” In: Virology, Fields et al. (eds.), New York: Raven Press, pp. 1437-1500, 1990.

Couch et al., Am. Rev. Resp. Dis., 88:394-403, 1963.

Coupar et al., Gene, 68:1-10, 1988.

Curiel, “Gene transfer mediated by adenovirus-polylysine DNA complexes,” In: Viruses in Human Gene Therapy, J. -M. H. Vos (Ed.), Carolina Academic Press, Durham, N.C., pp 179-212, 1994.

EPO 0273085.

Flotte et al., Gene Therapy, 2:29-37, 1995.

Fodor et al., Nature 364(6437):555-6, 1995.

Forster and Symons, Cell, 49:211-220, 1987.

Fraley and Fornari Kaplan, Proc. Nat'l. Acad. Sci. USA 76:3348-3352, 1979.

Friedmann, “Progress toward human gene therapy,” Science, 244:1275-1281, 1989.

Gaffney et al., Proc Nat'l Acad Sci USA 95:14875-14879, 1998.

Gerlach et al., Nature, 328:802-805, 1987.

Ghosh and Bachhawat, “Targeting of liposomes to hepatocytes,” In: Liver Diseases, Targeted Diagnosis and Therapy Using Specific Receptors and Ligands, Wu and Wu (Eds.), Marcel Dekker, New York, pp 87-104, 1991.

Ghosh-Choudhury et al., EMBO J., 6:1733-1739, 1987.

Glynne et al., Curr Opin Immunol 12:210-214, 2000.

Gomez-Foix et al., J. Biol. Chem., 267:25129-25134, 1992.

Graham et al., J. Gen. Virol., 36:59-72, 1977.

Graham and Prevec, Biotechnology, 20:363-390, 1992.

Grunhaus and Horwitz, Seminar in Virology, 3:237-252, 1992.

Harley et al Curr. Op. Immun., 10:690-696, 1998.

Haseloff and Gerlach, Nature, 334:585-591, 1988.

Hersdorffer et al., DNA Cell Biol., 9:713-723, 1990.

Hermonat and Muzyczka, Proc. Nat'l. Acad. Sci. USA, 81:6466-6470, 1984.

Herz and Gerard, “Proc. Nat'l. Acad. Sci. USA 90:2812-2816, 1993.

Horwich et al., J. Virol., 64(2):642-50, 1990.

Jones and Shenk, Cell, 13:181-188, 1978.

Joyce, Nature, 338:217-244, 1989.

Kaneda et al., Science, 243:375-378, 1989.

Kaplitt et al., Nature Genetics, 8:148-154, 1994.

Karlsson et al., EMBO J., 5:2377-2385, 1986.

Kasahara et al., Science, 266:1373-6, 1994.

Kato et al., J. Biol. Chem., 266:3361-3364, 1991.

Kim and Cech, Proc. Nat'l Acad. Sci. USA, 84:8788-8792, 1987.

Klein et al., Nature, 327:70-73, 1987.

Kotin et al., Proc. Nat'l Acad. Sci. USA, 87:2211-2215, 1990.

Laughlin et al., J. Virol., 60:515-524, 1986.

LaFace et al., Viology, 162:483-486, 1988.

Lebkowski et al., Mol. Cell. Biol., 8:3988-3996, 1988.

Le Gal La Salle et al., Science, 259:988-990, 1993.

Levrero et al., Gene, 101: 195-202, 1991.

Lieber and Strauss, Mol. Cell. Biol., 15: 540-551, 1995.

Mann et al., Cell, 33:153-159, 1983.

Markowitzetal., J. Virol., 62:1120-1124,1988.

McCarty et al., J. Virol., 65:2936-2945, 1991.

McLaughlin et al., J. Virol., 62:1963-1973, 1988.

Michel and Westhof, J. Mol. Biol., 216:585-610, 1990.

Miller, Curr. Top. Microbiol. Immunol., 158:1, 1992.

Mohan et al., J. Immunol. 159:454-465, 1997.

Mohan et al., J. Clin. Invest. 103:1685-1695, 1999.

Mohan et al., J. Immunol., 162:6492-6502, 1999.

Mohan et al., J. Clin. Invest. 101:1362-1372, 1998.

Mohan et al., 1998. Genetic insights into murine lupus. In Lupus: Molecular and Cellular Pathogenesis. G. Kammer and G. C. Tsokos, editors. Humana Press, Totowa, N.J. 124-139.

Mohan et al., J. Immunol., 159:454-465, 1997.

Morel et al., J. Immunol., 158:6019-6028, 1997.

Morel et al., Mamm. Genome, 10:176-181, 1999.

Morel et al., Mamm. Genome, 7:335-9, 1996.

Morel et al., Immun, 11:131-139, 1999.

Moser et al., Proc Nat'l Acad Sci USA, 95:14869-14874, 1998.

Muzyczka, Curr. Top. Microbiol. Immunol., 158:97-129, 1992.

Nicolas and Rubinstein, “Retroviral vectors,” In: Vectors: A survey of molecular cloning vectors and their uses, Rodriguez and Denhardt (eds.), Stoneham: Butterworth, pp. 494-513, 1988.Temin, 1986.

Nicolau and Sene, Biochem. Biophys. Acta, 721:185-190, 1982.

Nicolau et al., Methods Enzymol., 149:157-176, 1987.

Ohi et al., Gene, 89L:279-282, 1990.

Palukaitis et al., Virology, 99:145-151, 1979.

Paskind et al., Virology, 67:242-248, 1975.

Perales et al., Proc. Nat'l Acad. Sci. USA, 91:4086-4090, 1994.

Perriman et al., Gene, 113:157-163, 1992.

Probst et al., Science, 280:1447-1451, 1998.

Prody et al., Science, 231:1577-1580, 1986.

Renan, Radiother. Oncol., 19:197-218, 1990.

Ragot et al., Nature, 361:647-650, 1993.

Racher et al., Biotechnology Techniques, 9:169-174, 1995.

Reinhold-Hurek and Shub, Nature, 357:173-176, 1992.

Remington's Pharmaceutical Sciences, 15th Edition, Chapter 61, pages 1035-1038 and 1570-1580.

Rich et al., Hum. Gene Ther., 4:461-476, 1993.

Ridgeway, “Mammalian expression vectors,” In: Vectors: A survey of molecular cloning vectors and their uses. Rodriguez and Denhardt, eds. Stoneham: Butterworth, pp. 467-492, 1988.

Rosenfeld et al., Science, 252:431-434, 1991.

Rosenfeld et al., Cell, 68:143-155, 1992.

Roux et al., Proc. Nat'l Acad. Sci. USA, 86:9079-9083, 1989.

Samulski et al., J. Virol., 63:3822-3828, 1989.

Samulski et al., EMBO J., 10:3941-3950, 1991.

Sarver et al., Science, 247:1222-1225, 1990.

Scanlon et al., Proc Nat'l Acad Sci USA, 88:10591-10595, 1991.

Shai et al., Human Molecular Genetics, 8:639-644, 1999.

Shelling and Smith, Gene Therapy, 1:165-169, 1994.

Sioud et al., J Mol. Biol., 223:831-835, 1992.

Stratford-Perricaudet and Perricaudet., In: Human Gene Transfer, Eds, Cohen-Haguenauer and Boiron, Editions John Libbey Eurotext, France, pp. 51-61, 1991.

Stratford-Perricaudet et al., Hum. Gene. Ther., 1:241-256, 1991.

Symons, Annu. Rev. Biochem., 61:641-671, 1992.

Symons, Nucl. Acids Res., 9:6527-6537, 1981.

Temin, Cell Biophys., 9:9-16, 1986.

Thompson et al., Nature Genet., 9:444-450, 1995.

Top et al., J. Infect. Dis., 124:155-160, 1971.

Tratschin et al., Mol. Cell. Biol., 4:2072-2081, 1984.

Tsao et al., J. Clin. Invest. 99:725-731, 1997.

U.S. Pat. No. 6,225,322.

U.S. Pat. No. 6,218,406.

U.S. Pat. No. 6,218,401.

U.S. Pat. No. 6,214,828.

U.S. Pat. No. 6,211,193.

U.S. Pat. No. 6,187,786.

U.S. Pat. No. 6,177,432.

U.S. Pat. No. 6,169,096.

U.S. Pat. No. 6,159,984.

U.S. Pat. No. 6,143,766.

U.S. Pat. No. 6,143,758.

U.S. Pat. No. 6,127,366.

U.S. Pat. No. 6,124,465.

U.S. Pat. No. 6,124,295.

U.S. Pat. No. 6,103,723.

U.S. Pat. No. 6,093,737.

U.S. Pat. No. 6,090,948.

U.S. Pat. No. 6,080,870.

U.S. Pat. No. 6,077,853.

U.S. Pat. No. 6,071,907.

U.S. Pat. No. 6,066,738.

U.S. Pat. No. 6,063,930.

U.S. Pat. No. 6,060,038.

U.S. Pat. No. 6,054,466.

U.S. Pat. No. 6,051,582.

U.S. Pat. No. 6,051,574.

U.S. Pat. No. 6,051,565.

U.S. Pat. No. 6,040,305.

U.S. Pat. No. 6,037,350.

U.S. Pat. No. 6,030,982.

U.S. Pat. No. 6,028,201.

U.S. Pat. No. 6,015,817.

U.S. Pat. No. 6,013,662.

U.S. Pat. No. 6,011,029.

U.S. Pat. No. 6,001,835.

U.S. Pat. No. 5,998,407.

U.S. Pat. No. 5,990,277.

U.S. Pat. No. 5,985,879.

U.S. Pat. No. 5,981,562.

U.S. Pat. No. 5,977,134.

U.S. Pat. No. 5,976,851.

U.S. Pat. No. 5,972,984.

U.S. Pat. No. 5,972,966.

U.S. Pat. No. 5,972,942.

U.S. Pat. No. 5,968,965.

U.S. Pat. No. 5,968,952.

U.S. Pat. No. 5,965,578.

U.S. Pat. No. 5,965,570.

U.S. Pat. No. 5,958,940.

U.S. Pat. No. 5,945,430.

U.S. Pat. No. 5,945,429.

U.S. Pat. No. 5,939,557.

U.S. Pat. No. 5,939,439.

U.S. Pat. No. 5,936,097.

U.S. Pat. No. 5,932,590.

U.S. Pat. No. 5,929,077.

U.S. Pat. No. 5,925,651.

U.S. Pat. No. 5,925,648.

U.S. Pat. No. 5,922,883.

U.S. Pat. No. 5,919,785.

U.S. Pat. No. 5,919,780.

U.S. Pat. No. 5,914,341.

U.S. Pat. No. 5,891,889.

U.S. Pat. No. 5,889,053.

U.S. Pat. No. 5,885,995.

U.S. Pat. No. 5,883,105.

U.S. Pat. No. 5,880,140.

U.S. Pat. No. 5,874,452.

U.S. Pat. No. 5,872,136.

U.S. Pat. No. 5,872,135.

U.S. Pat. No. 5,869,682.

U.S. Pat. No. 5,861,395.

U.S. Pat. No. 5,859,015.

U.S. Pat. No. 5,859,012.

U.S. Pat. No. 5,856,439.

U.S. Pat. No. 5,856,326.

U.S. Pat. No. 5,854,265.

U.S. Pat. No. 5,854,264.

U.S. Pat. No. 5,852,010.

U.S. Pat. No. 5,817,678.

U.S. Pat. No. 5,789,438.

U.S. Pat. No. 5,780,492.

U.S. Pat. No. 5,780,488.

U.S. Pat. No. 5,756,528.

U.S. Pat. No. 5,753,650.

U.S. Pat. No. 5,750,567.

U.S. Pat. No. 5,736,539.

U.S. Pat. No. 5,710,171.

U.S. Pat. No. 5,703,241.

U.S. Pat. No. 5,703,067.

U.S. Pat. No. 5,686,472.

U.S. Pat. No. 5,663,193.

U.S. Pat. No. 5,661,161.

U.S. Pat. No. 5,661,128.

U.S. Pat. No. 5,652,257.

U.S. Pat. No. 5,631,280.

U.S. Pat. No. 5,627,202.

U.S. Pat. No. 5,627,057.

U.S. Pat. No. 5,624,936.

U.S. Pat. No. 5,585,359.

U.S. Pat. No. 5,427,910.

U.S. Pat. No. 5,523,207.

U.S. Pat. No. 5,837,832.

U.S. Pat. No. 5,837,860.

U.S. Pat. No. 5,861,242.

U.S. Pat. No. 5,578,832.

U.S. Pat. No. 5,139,941.

U.S. Pat. No. 5,578,629.

U.S. Pat. No. 5,576,313.

U.S. Pat. No. 5,576,293.

U.S. Pat. No. 5,571,835.

U.S. Pat. No. 5,567,729.

U.S. Pat. No. 5,536,750.

U.S. Pat. No. 5,534,537.

U.S. Pat. No. 5,523,456.

U.S. Pat. No. 5,523,430.

U.S. Pat. No. 5,510,510.

U.S. Pat. No. 5,510,371.

U.S. Pat. No. 5,504,212.

U.S. Pat. No. 5,504,115.

U.S. Pat. No. 5,491,164.

U.S. Pat. No. 5,354,855.

U.S. Pat. No. 5,168,053.

U.S. Pat. No. 5,624,824.

U.S. Pat. No. 5,625,047.

U.S. Pat. No. 5,614,396.

U.S. Pat. No. 5,624,830.

U.S. Pat. No. 5,480,893.

U.S. Pat. No. 5,468,733.

U.S. Pat. No. 5,439,918.

U.S. Pat. No. 5,436,263.

U.S. Pat. No. 5,420,334.

U.S. Pat. No. 5,420,245.

U.S. Pat. No. 5,420,157.

U.S. Pat. No. 5,352,705.

U.S. Pat. No. 5,350,867.

U.S. Pat. No. 5,340,828.

U.S. Pat. No. 5,326,773.

U.S. Pat. No. 5,260,479.

U.S. Pat. No. 5,260,465.

U.S. Pat. No. 5,245,061.

U.S. Pat. No. 5,238,922.

U.S. Pat. No. 5,185,248.

U.S. Pat. No. 5,580,729.

U.S. Pat. No. 4,683,195.

U.S. Pat. No. 4,683,202.

U.S. Pat. No. 4,683,194.

U.S. Pat. No. 4,797,368.

U.S. Pat. No. 4,430,434.

U.S. Pat. No. 4,727,028.

U.S. Pat. No. 4,960,704.

U.S. Pat. No. 4,559,302.

U.S. Pat. No. 4,608,251.

U.S. Pat. No. 4,601,903.

U.S. Pat. No. 4,599,231.

U.S. Pat. No. 4,599,230.

U.S. Pat. No. 4,596,792.

U.S. Pat. No. 4,578,770.

Wagner et al., Science, 260:1510-1513, 1990.

Wakeland et al.

Journal of Clinical Immunology 17:272-281, 1997.

Walsh et al., J. Clin. Invest., 94:1440-1448, 1994.

Wei et al., Gene Therapy, 1:261-268, 1994.

Winchester, Genetic susceptibility to systemic lupus erythematosus. In Systemic lupus erythematosus. R. G. Lahita, editor. Churchill Livingstone, New York. 65-85, 1992.

Wong et al., Gene, 10:87-94, 1980.

Wu and Wu, J. Biol. Chem., 262:4429-4432, 1987.

Wu and Wu, Adv. Drug Delivery Rev., 12:159-167, 1993.

Yang et al., Proc. Nat'l Acad. Sci. USA, 87:9568-9572, 1990.

Yuan and Altman, Science, 263:1269-1273, 1994.

Yuan et al., Proc. Nat'l Acad. Sci. USA, 89:8006-8010, 1992.

Yoder et al., Blood, 82 (Supp.): 1:347A, 1994.

Zhou et al., Exp. Hematol. (NY), 21:928-933, 1993.

Zhou et al., J. Exp. Med., 179:1867-1875, 1994.

What is claimed is:

1. A method for treating systemic lupus erythematosus (SLE) comprising administering to a subject suffering from SLE a first inhibitor of farnesyl-protein transferase (FPT).

2. The method of claim 1, wherein said inhibitor is administered through intravenous, intraarterial, intramuscular, intraperitonical, intradermal, intranasal, oral or topical routes.

3. The method of claim 1, wherein said inhibitor is a pharmaceutical compound.

4. The method of claim 3, wherein said pharmaceutical compound is selected from the group consisting of a tricyclic, a quinolinone, a quinazolinone, a benzopyranone, an imidazole, a benzo(F)isoindole, a thioproline, a biheteroaryl, a piperazinyl, and a piperazinonyl.

5. The method of claim 1, wherein said inhibitor is a farnesyl pyrophosphate analogue.

6. The method of claim 1, wherein said inhibitor is a peptide or peptidomimetic.

7. The method of claim 6, wherein said peptide or peptidomimetic contains or emulates the CAAX-box motif.

8. The method of claim 1, wherein said inhibitor is a nucleic acid encoding an FPT antisense molecule or an FPT ribozyme under the control of a promoter active in said subject.

9. The method of claim 8, wherein said nucleic acid is contained in a vector.

10. The method of claim 9, wherein said vector is a viral vector.

11. The method of claim 10, wherein said viral vector is selected from the group consisting of an adenoviral vector, an adeno-associated viral vector, a retroviral vector, a vaccinia viral vector, a herpesviral vector and a polyoma viral vector.

12. The method of claim 8, wherein said antisense molecule targets an FPT promoter, intron, transcription start site, translation start site, splice junction or coding region.

13. The method of claim 1, wherein said inhibitor is encapsulated in a liposome.

14. The method of claim 1, wherein said inhibitor is administered at least a second time.

15. The method of claim 14, wherein said inhibitor is administered as part of a continuous maintenance drug regimen.

16. The method of claim 1, wherein said subject is monitored for toxic effects following administration of said inhibitor.

17. The method of claim 1, wherein said cells from subject said are monitored for FPT activity following administration of said inhibitor.

18. The method of claim 1, further comprising administering to said subject a second inhibitor of FPT distinct from said first inhibitor.

19. The method of claim 1, further comprising administering to said subject a conventional SLE therapeutic compound.

20. The method of claim 19, wherein said conventional SLE therapeutic compound is administered prior to said inhibitor.

21. The method of claim 19, wherein said conventional SLE therapeutic compound is administered after said inhibitor.

22. The method of claim 19, wherein said conventional SLE therapeutic compound is administered at the same time as said inhibitor.

23. The method of claim 19, wherein said conventional SLE therapeutic compound is administered at least a second time.

24. The method of claim 19, wherein said conventional SLE therapeutic compound is administered as part of a continuous maintenance drug regimen.

25. The method of claim 19, wherein said conventional SLE therapeutic compound is selected from the group consisting of a non-steroidal anti-inflammatory drug (NSAID), an antimalarial drug, a corticosteroid hormone and an immunosuppressive drug.

26. The method of claim 25, wherein said NSAID is selected from the group consisting of ibuprofen, naproxen, sulindac, diclofenac, piroxicam, ketoprofen, diflunisal, nabumetone, etodolac, oxaprozin and indomethacin.

27. The method of claim 25, wherein said antimalarial drug is selected from the group consisting of hydrochloroquine, chloroquine and quinacrine.

28. The method of claim 25, wherein said corticosteroid hormone is selected from the group consisting of prednisone, hydrocortisone, methylprednisone and dexamethasone.

29. The method of claim 25, wherein said immunosuppressive agent is selected from the group consisting of azathioprine, cyclophosphamide, and methotrexate.

30. The method of claim 19, wherein said subject is monitored for toxic effects following administration of said conventional SLE therapeutic compound.
